(19) United States
(12) Patent Application Publication
(10) Pub. No.: US 2023/0346104 A1
(43) Pub. Date: Nov. 2, 2023

(54) APPLICATOR WITH CARTRIDGES CONFIGURED TO DELIVER PLASMA ENERGIES FOR SKIN TREATMENT

(71) Applicant: L'Oreal, Paris (FR)

(72) Inventors: Gregoire CHARRAUD, Jersey City, NJ (US); Casey BARBARINO, San Anselmo, CA (US); Rafael FELICIANO, New Providence, NJ (US)

(73) Assignee: L'Oreal, Paris (FR)

(21) Appl. No.: 17/733,200

(22) Filed: Apr. 29, 2022

(51) Int. Cl. A61N 5/06; H05H 1/24; A45D 34/04; A61N 1/44

(52) U.S. Cl. CPC A45D 34/041; A61N 1/44; A61N 5/0616; H05H 1/24; A61N 2005/0652; A61N 2005/0658; H05H 2245/34

(57) ABSTRACT

A skin care system including a dispensing device configured to administer a light therapy and a plasma treatment, and an applicator configured to apply a formula, comprising a reservoir configured to hold the formula, a roller ball configured to apply the formula, an attachment configured to attach the cartridge to the dispensing device, and a microcontroller configured to direct the dispensing devices to apply the plasma treatment.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, a skin care system including a dispensing device configured to administer a light therapy and a plasma treatment, and an applicator configured to apply a formula, including a reservoir configured to hold the formula, a roller ball configured to apply the formula, an attachment configured to attach the cartridge to the dispensing device, and a microcontroller configured to direct the dispensing device to apply the plasma treatment is disclosed.

In another aspect, a method of administering multiple skin treatments, using the skin care system, the method including selecting an applicator filled with a formula, placing the applicator into the dispensing device, identifying a plasma treatment to apply based on the applicator, applying the formula, administering light therapy, and administering the plasma treatment is disclosed.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is an example applicator, in accordance with the present technology;

FIG. 2 is an example cross-section of an applicator, in accordance with the present technology;

FIG. 3 is an example dispensing device, in accordance with the present technology; and

FIG. 4 is an example method of using a skin care system, in accordance with the present technology.

### DETAILED DESCRIPTION

While illustrative embodiments have been illustrated and described, it will be appreciated that various changes can be made therein without departing from the spirit and scope of the invention.

Described herein is an applicator with a microcontroller that is capable of instructing a dispensing device to administer a plasma treatment while applying both formula and light therapy. In some embodiments, the plasma treatment is a cold plasma treatment. In some embodiments, the plasma treatment is administered through an electrode and one or more wires. In some embodiments, the dispensing device applies high voltage through the one or more wires to the electrode to generate plasma as a roller ball on the applicator rolls over a surface.

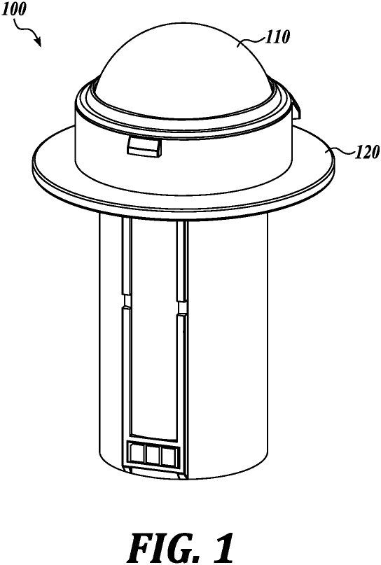

FIG. 1 is an example applicator, in accordance with the present technology. The applicator 100 may include a roller ball 110, and an attachment 120.

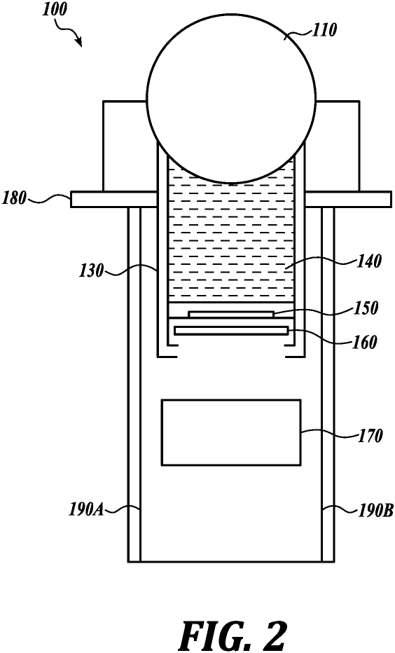

The roller ball 110 may be configured to distribute and apply a formula located a reservoir inside the applicator 100 (as shown in FIG. 2). In some embodiments, the roller ball 110 is plastic, but in other embodiments, the roller ball 110 may be glass or metal.

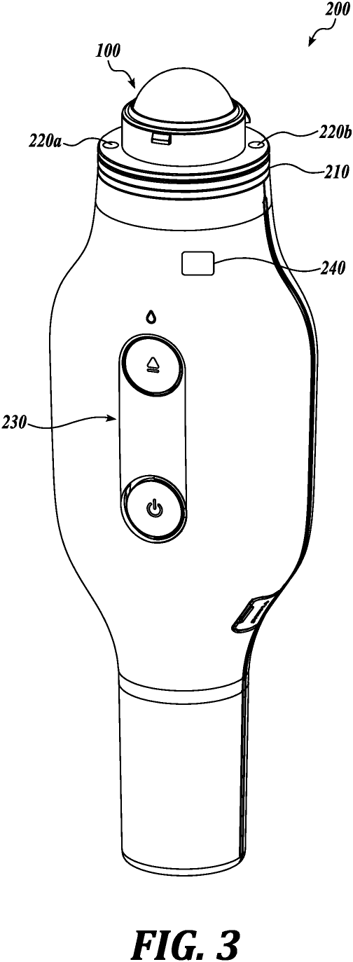

In some embodiments, the applicator 100 also includes an attachment 120 configured to secure the applicator 100 into a dispensing device, such as the dispensing device 200 in FIG. 3. While the attachment 120 is illustrated as a disk shaped to couple to a dispensing device, the attachment 120 may take any form capable of securing the applicator to a dispensing device including a threaded attachment, a magnet, or an attachment configured to snap into the dispensing device. In some embodiments, the attachment 120 is clear so that the dispensing device is visible through the attachment.

In operation, the applicator 100 can be placed inside a dispensing device (as shown in FIG. 3) and secured to the dispensing device with the attachment 120. The roller ball 110 can be rolled over a surface, such as a user’s skin, to apply a formula.

FIG. 2 is an example cross-section of an applicator 100, in accordance with the present technology. The applicator 100 may include a roller ball 110, an attachment 120, a reservoir 130 configured to hold a formula 140, a piston 150, a microcontroller 160, and a processor 170. In some embodiments, the applicator further includes an electrode 180, and one or more wires, 190a, 190b.

In some embodiments, the reservoir 130 is located inside the applicator 100, and is configured to hold a formula 140. In some embodiments, the formula 140 is a skin care formula. In some embodiments, the skin care formula is a moisturizer, a toner, an acne treatment, a wrinkle treatment, fine line treatment, or a cosmetic. As the roller ball 110 rolls, formula 140 from the reservoir 130 is applied to a surface.

In some embodiments, the applicator 100 further includes a piston 150 configured to push the formula 140 towards the roller ball 110 as the formula is applied. In some embodiments, the piston 150 is directed by circuitry on a dispensing device or on the applicator itself to push the formula 140.

In some embodiments, the applicator 100 includes a microcontroller 160 configured to identify the type of formula 140 inside the applicator 100 to a dispensing device. In some embodiments, the microcontroller 160 further identifies the type of treatment the applicator should apply, i.e., the type of plasma treatment that should be applied with the type of formula 140. In some embodiments, the plasma treatment is cold plasma treatment. In some embodiments, the plasma treatment is continuous. In some embodiments, the plasma treatment is pulsed. In some embodiments, the plasma treatment is applied for a specific time amount, such as one minute, or five minutes. In some embodiments, the applicator stops administering plasma treatment when the specific time amount has elapsed. The microcontroller 160 may also be used to identify any number of things about the formula 140 or applicator 100, including the amount of formula 140 inside the applicator 100, the expiration date of the formula 140 inside the applicator 100, or when to replace the applicator 100.

In some embodiments, the applicator 100 further includes an electrode 180, and one or more wires 190A, 190B configured to administer a plasma treatment. In some embodiments, the electrode 180 is the attachment 120. In some embodiments, the electrode 180 is made of a conductive material. In some embodiments, the electrode 180 is located inside the attachment 120 (as shown in FIG. 1) and is merely a component of the attachment 120. In some embodiments, one or more wires 190A, 190B connect to the electrode 180 to generate the plasma. In some embodiments, high voltage is applied to the one or more wires 190A, 190B by the applicator. In some embodiments, high voltage is applied to the one or more wires 190A, 190B from a dispensing device (such as dispensing device 200 in FIG. 3). In some embodiments, the high voltage is above 25 kV. In some embodiments, the high voltage is above 50 kV. In operation, high voltage can be applied to the one or more wires 190A, 190B and to the electrode 180 to generate plasma while the user rolls the applicator 100 on their skin.

FIG. 3 is an example dispensing device, in accordance with the present technology. In some embodiments, the applicator 100 can be attached to a dispensing device 200. In some embodiments, the dispensing device includes an end 210, one or more light sources 220a, 220b, an actuator 230, and a contactless reader 240. In some embodiments, the applicator 100 connects to the dispensing device 200.

In some embodiments, the dispensing device 200 includes an end 210. The end 210 may be configured to be seen through the attachment 120 on the applicator 100. In some embodiments, the end 210 includes one or more light sources 220a, 220b configured to administer light therapy to a surface while the formula 140 is being applied.

In some embodiments, the one or more light sources 220a, 220b are LEDs. In some embodiments, there are only two light sources 220a, 220b on the dispensing device. In some embodiments, a first light source 220a is configured to administer light therapy in a first wavelength. In some embodiments, a second light source 220b is configured to administer light therapy in a second wavelength. In some embodiments, the light therapy in the first wavelength and the light therapy in the second wavelength are administered simultaneously. In some embodiments, the light therapy and applying the formula happen simultaneously. In some embodiments, there are one or more light sources 220a, 220b in a ring, located around the end 210 of the dispensing device 200.

In some embodiments, the dispensing device 200 includes one or more actuators 230. While the actuator 230 is illustrated as a button, in some embodiments, the actuator 230 may be a switch, a capacitive touch type button, a dial, or the like. The actuator 230 may be configured to begin the administration of light therapy, to apply the formula, to control the plasma treatment, or all three. In some embodiments, the dispensing device 200 also includes a contact-less chip reader 240 to read the microcontroller 160 on the applicator 100.

In operation, a user may place an applicator 100 into the dispensing device 200. When the actuator 230 is actuated, the formula is applied, the light therapy is administered, or both, simultaneously. A user may then apply the formula with the applicator 100. Additionally, the plasma treatment can be administered at the same time as the light therapy and applying the formula.

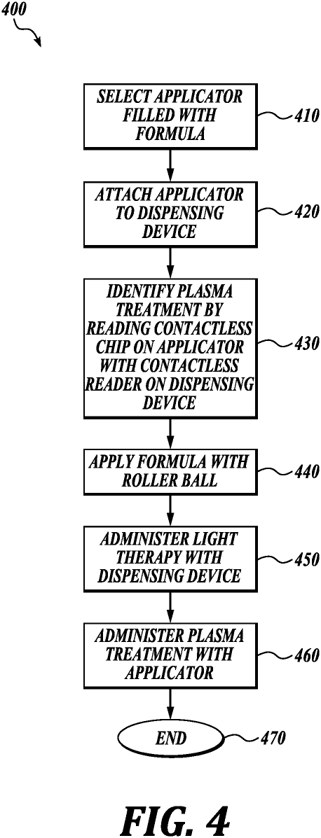

FIG. 4 is an example method 400 of using a skin care system, in accordance with the present technology.

In block 410, an applicator is selected having a specific formula and configured to direct the applicator and/or the dispensing device to administer a specific plasma treatment. In some embodiments, the plasma treatment is cold plasma treatment. In some embodiments, the plasma treatment is continuous. In some embodiments, the plasma treatment is pulsed. In some embodiments, the plasma treatment is applied for a specific time amount, such as one minute, or five minutes. In some embodiments, the applicator stops administering plasma treatment when the specific time amount has elapsed. In some embodiments, the applicator further directs the applicator and/or the dispensing device to administer the plasma treatment at a specific voltage.

In block 420, the applicator is attached to the dispensing device. In some embodiments, the applicator is attached to the dispensing device with an attachment. In some embodiments, the applicator is clear to allow one or more light sources on the dispensing device to emit light through the attachment. In some embodiments, the applicator slides, clicks, or connects into the dispensing device. In some embodiments, the applicator attaches to the dispensing device with a threaded connection or a magnet.

In block 430, the dispensing device reads the microprocessor (or contactless chip) on the applicator with a contactless reader. In some embodiments, the microprocessor on the applicator identifies the type of formula, the type of plasma treatment to administer, the lifetime of the applicator, the amount of formula inside the applicator, or a combination thereof.

In block 440, formula is applied as the roller ball is rolled along a surface. In some embodiments, the surface is a user’s face. In some embodiments, the surface may be any portion of the user’s skin.

In block 450, light therapy is applied with the dispensing device. In some embodiments, the dispensing device includes an actuator configured to begin both the application of the formula and the administration of the light therapy. In some embodiments, the dispensing device includes two or more light sources configured to administer the light therapy. In some embodiments, the dispensing device is configured to deliver two or more types of light therapy, either one at a time, or simultaneously. In such embodiments, one light source is configured to administer a first light therapy, and another light source is configured to administer a second light therapy. In some embodiments, the first light therapy may include emitting a first light at a first wavelength, and the second light therapy may include emitting a second light at a second wavelength.

In block 460, the plasma treatment is applied with the applicator. In some embodiments, blocks 440, 450, and 460 occur simultaneously.

In block 470, the method ends.

1. A skin care system comprising:
a dispensing device configured to administer a light therapy and a plasma treatment; and
an applicator configured to apply a formula, comprising:
a reservoir configured to hold the formula,
a roller ball configured to apply the formula,
an attachment configured to attach the cartridge to the dispensing device, and
a microcontroller configured to direct the dispensing device to apply the plasma treatment.

2. The skin care system of claim 1, wherein the light therapy and the plasma treatment are administered simultaneously with the application of the formula.

3. The skin care system of claim 1, wherein the dispensing device is configured to apply two or more wavelengths of light therapy simultaneously.

4. The skin care system of claim 1, wherein the dispensing device administers light therapy through one or more light sources.

5. The skin care system of claim 4, wherein the one or more light sources are a ring of LEDs around a top of the dispensing device.

6. The skin care system of claim 4, wherein the applicator further comprises a clear attachment so that the light from the one or more light sources on the dispensing device can pass through the attachment to administer the light therapy.

7. The skin care system of claim 7, wherein the applicator further comprises an electrode configured to create plasma on contact with a surface.

8. The skin care system of claim 7, wherein the attachment is the electrode.

9. The skin care system of claim 7, wherein the applicator further comprises a wire connecting the electrode to the dispensing device.

10. The skin care system of claim 9, wherein the dispensing device is further configured to pass high voltage through the wire and to the electrode.

11. A method of administering multiple skin treatments, using the device of claim 1, the method comprising:
selecting an applicator filled with a formula;
placing the applicator into the dispensing device;
identifying a plasma treatment to apply based on the applicator;
applying the formula;
administering light therapy; and
administering the plasma treatment.

12. The method of claim 11, wherein applying of the formula, the administering the light therapy, and administering the plasma treatment is done simultaneously.

13. The method of claim 11, wherein the dispensing device administers light therapy through one or more light sources.

14. The method of claim 13, wherein the one or more light sources are a ring of LEDs around a top of the dispensing device.

15. The method of claim 11, wherein the method further comprises identifying the formula in the applicator with the dispensing device.

16. The method of claim 11, wherein the method further comprises removing the applicator and placing a second applicator into the dispensing device to administer a second plasma treatment and/or a second formula.

17. The method of claim 11, wherein the method further comprises applying high voltage to an electrode in the applicator to administer plasma treatment.

18. The method of claim 17, wherein the high voltage is applied to the electrode with a dispensing device through a wire in the applicator.

19. The method of claim 11, wherein the method further comprises applying the plasma treatment for a specific time amount based on the microcontroller of the applicator.

20. The method of claim 11, wherein the method further comprises stopping the plasma treatment when the specific time amount has elapsed.
